(19) United States
(12) Patent Application Publication
(10) Pub. No.: US 2022/0220544 A1
(43) Pub. Date: Jul. 14, 2022

(54) SPATIAL NUCLEIC ACID DETECTION USING OLIGONUCLEOTIDE MICROARRAYS

(71) Applicant: AGILENT TECHNOLOGIES, INC., Santa Clara, CA (US)

(72) Inventors: Robert A. ACH, San Francisco, CA (US); Nicholas M. SAMPAS, San Jose, CA (US); Brian Jon PETER, Los Altos, CA (US)

(73) Assignee: AGILENT TECHNOLOGIES, INC., Santa Clara, CA (US)

(21) Appl. No.: 17/571,347

(22) Filed: Jan. 7, 2022

Related U.S. Application Data
(60) Provisional application No. 63/135,254, filed on Jan. 8, 2021.

(51) Int. Cl. C12Q 1/6837; C12Q 1/6806; C12Q 1/6876; C12Q 1/6816; C12Q 1/6874; C12N 15/10

(52) U.S. Cl. CPC C12Q 1/6837; C12Q 1/6806; C12N 15/1065; C12Q 1/6816; C12Q 1/6874; C12Q 1/6876

(57) ABSTRACT

The present disclosure is generally directed to detecting nucleic acids. In particular, disclosed herein are methods and compositions for determining the sequence (or identity) and location of RNA and other molecules in situ. The present invention is generally related to a method for detecting nucleic acids, the method including providing a tissue sample; providing an array comprising a plurality of oligonucleotide probes attached to a surface of the array, in which each oligonucleotide probe, of the plurality of oligonucleotide probes, includes a location barcode sequence, a primer binding sequence, and a priming sequence; releasing the plurality of oligonucleotide probes from the array surface; contacting the tissue sample with the released oligonucleotide probes; and allowing the released oligonucleotide probes to diffuse into the tissue sample.

### RELATED APPLICATION

This application claims priority to U.S. Provisional Application No. 63/135,254, filed Jan. 8, 2021, the entire disclosure of which is hereby incorporated by reference.

### SUBMISSION OF SEQUENCE LISTING ON ASCII TEXT FILE

This application contains a Sequence Listing submitted via EFS-Web, which is hereby incorporated by reference in its entirety. The ASCII text copy was created on, was named______.txt and is______bytes in size.

### FIELD OF THE INVENTION

The present disclosure relates generally to detecting nucleic acids. In particular, the present disclosure relates to methods and compositions for determining the sequence (or identity) and location of RNA and other molecules in situ. For example, there is disclosed a method for detecting nucleic acids, the method comprising providing a tissue sample; providing an array comprising a plurality of oligonucleotide probes attached to a surface of the array, wherein each oligonucleotide probe, of the plurality of oligonucleotide probes, comprises a location barcode sequence, a primer binding sequence, and a priming sequence; releasing the plurality of oligonucleotide probes from the array surface; contacting the tissue sample with the released oligonucleotide probes; and allowing the released oligonucleotide probes to diffuse into the tissue sample.

### BACKGROUND

Most current techniques for the analysis of gene expression patterns either provide spatial transcriptional information only for one or a handful of genes at a time, such as RNA Fluorescent in situ Hybridization (RNA FISH), or offer transcriptional information for many or all of the genes in a sample at the cost of losing positional information (such as RNA-sequencing or array analysis of gene expression).

Spatial RNA-sequencing, also known as spatial transcriptomics, is a recently developed technology used to spatially resolve RNA sequence data, and thereby obtain localized gene expression data from RNAs in individual tissue sections. A method for spatial transcriptomics was originally developed by Stahl, Lundeberg, and colleagues (Science 353, no. 6294 (2016): 78-82 and US Patent Application No. 2014/0066318 A1). Other variations of spatial RNA sequencing have been described in U.S. Pat. No. 9,371,598 B2 and US Patent Application No. 2018/0245142 A1. Also, a version of spatial RNA-sequencing (Nature Protocols 13, (2018): 2501-2534) is now offered commercially. In this method, spatially-barcoded reverse transcription oligo (dT) primers are attached to the surface of a microscope slide at their 5′ ends in an ordered manner. A tissue cryosection is then mounted atop this microscope slide, and the tissue is then permeabilized to cause the release of the RNA so that the barcoded primers can bind to the mRNAs from the tissue. The barcoded primers are then used to initiate reverse transcription of the bound mRNA, and the resulting cDNAs thus incorporate the spatial barcodes of the primers. Sequencing libraries are then prepared from the resulting cDNAs and analyzed by DNA sequencing. The spatial barcode present within each generated sequence allows the data for each individual mRNA transcript to be mapped back to its point of origin on the array, and thus within the tissue section. A major disadvantage of this technique, and the other methods described in the patent applications above, is that these methods cannot be used on formalin-fixed paraffin-embedded (FFPE) tissues, as the RNA in these tissues is cross-linked to the tissue, and thus cannot be released by permeabilization. Also, FFPE tissue sections are often already mounted on slides, and thus are unavailable to be mounted onto the array slide as an intact tissue section.

In general, the majority of previously described methods share common limitations: either they require nucleic acids to diffuse from the tissue to a solid support, and/or they require significant biochemical steps to occur on the solid support. An obvious drawback of these strategies is that the diffusion of the nucleic acids through tissue can be non-uniform, and hard to measure, making the true content of the tissue difficult to assess. In addition, while it is clear that biochemical processes, such as primer extension, can happen on a solid support, diffusion and mixing of reagents can be limited, leading to lower reaction efficiency. Also, the concentration of the solid-support bound element can be lower than optimal. Finally, previous methods for spatial RNA sequencing typically rely on a single sequence for initiation of cDNA synthesis, for example, an oligo-dT primer for initiation of cDNA synthesis of polyadenylated RNAs. However, this restricts the RNAs that can be measured to only those having a poly-A tail, which excludes many classes of RNAs and some messenger RNAs, and does not enable specific measurement of only a subset of RNAs of interest. Therefore, there remains a need for better methods to analyze gene expression information in the context of spatial information in a tissue section.

Accordingly, there exists a need for compositions and methods that allow for determining transcriptional information for many or all of the genes in a sample as well as determining positional information for these transcripts.

### BRIEF DESCRIPTION OF THE INVENTION

The present invention is generally related to a method for detecting nucleic acids, the method comprising providing a tissue sample; providing an array comprising a plurality of oligonucleotide probes attached to a surface of the array, wherein each oligonucleotide probe, of the plurality of oligonucleotide probes, comprises a location barcode sequence, a primer binding sequence, and a priming sequence; releasing the plurality of oligonucleotide probes from the array surface; contacting the tissue sample with the released oligonucleotide probes; and allowing the released oligonucleotide probes to diffuse into the tissue sample.

### BRIEF DESCRIPTION OF THE DRAWINGS

The disclosure will be better understood, and aspects and advantages other than those set forth above will become apparent, when consideration is given to the following detailed description thereof. Such detailed description makes reference to the following drawings, wherein:

FIG. 1A is a schematic illustrating the first step in an exemplary aspect wherein a plurality of oligonucleotides probes is attached to an array surface with a cleavable linker. FIG. 1B illustrates a related aspect wherein a plurality of first oligonucleotides hybridize to location barcodes of a plurality of oligonucleotide probes, which could be released. In both cases, each array feature (i.e., “location” on the array, or group of oligonucleotides located in a same location on the array) comprises a location barcode (shown as a patterned segment) that is distinct from location barcodes at other locations on the array.

FIG. 2 is a schematic illustrating an overview of a method of detecting nucleic acids in a tissue sample. The method can include releasing oligonucleotide probes from an array surface so that they remain in their respective location, applying a tissue slide so that the tissue is in contact with the released oligonucleotide probes on the array surface; and diffusing the released oligonucleotide probes into the tissue. The method includes synthesizing in situ first strand cDNA in the tissue section prior to applying the tissue slide.

FIG. 3 is a schematic illustrating the steps in an exemplary aspect wherein first-strand cDNA is made in situ, with use of a template-switching oligonucleotide (TSO), followed by use of spatially-barcoded oligonucleotides from a released oligonucleotide array to prime second strand cDNA synthesis.

FIG. 4 is a schematic illustrating an array of oligonucleotide probes containing an oligo(dT) primer region, according to another aspect of the invention.

FIG. 5 is a schematic illustrating the steps in an exemplary aspect wherein first-strand cDNA is primed by spatially-barcoded oligo(dT) primers from a cleaved oligonucleotide array. A template-switching oligonucleotide (TSO) is used to add a primer region to the 3′ end of the first strand cDNA, followed by second strand synthesis and PCR.

FIG. 6 is a schematic illustrating a method according to another aspect of the invention. An array of location-barcoded oligonucleotide probes hybridized to a library of oligonucleotides by base-paring with the spatial barcodes. The 3′ ends of the hybridized oligonucleotides contain an oligo(dT) region. A tissue slide can be brought into contact with the array, after the hybridized probes have been released, so that the released probes can diffuse into the tissue section, and can hybridize to the poly(A) tails of mRNAs in the tissue section. Synthesis of cDNA from these probes can generate nucleic acids comprising spatial barcodes and a copy of mRNA sequences from the tissue.

FIG. 7 is a schematic illustrating a first-strand cDNA, which was made in situ a method as shown in FIG. 6, is then ligated to an oligonucleotide containing a primer-binding sequence, allowing for primer binding and the subsequent second-strand cDNA synthesis and PCR.

FIG. 8A is a schematic illustrating a method according to another aspect of the invention in which spatially barcoded oligonucleotide probes can be used to detect the presence of oligonucleotide-tagged antibodies, as a proxy for protein expression. In particular, a tissue can be treated by a mixture of antibodies, wherein different antibodies can be tagged with oligonucleotides comprising different antibody index sequences. After unbound antibodies are washed away, the tissue section can be brought into contact with an array of releasable spatially-barcoded oligonucleotide probes. In FIG. 8B, the released oligonucleotide probes can diffuse into the tissue containing the oligonucleotide-linked antibodies. The 3′ ends of the hybridized oligonucleotides contain a region (dashed line) which can hybridize to a complementary sequence at the 3′ end of oligonucleotides linked to antibodies bound to various cellular targets in a tissue section. After primer extension, the oligonucleotide probes can copy the sequence of the oligonucleotide tags on the antibodies, including the antibody index sequences and the adjacent PCR primers. After amplification with the appropriate set of PCR primers, the resulting library of sequences can be used to profile spatially barcoded protein expression in the tissue section. In some aspects, this method of protein expression profiling can be combined with methods of RNA measurement as described elsewhere herein.

FIG. 9 is a schematic illustrating a method according to another aspect of the invention in which a set of oligonucleotide probes can hybridize to RNA in a tissue section on both sides of an exon-exon boundary. Ligation of the probes followed by PCR allows for detection of specific RNA species.

FIG. 10 is a schematic illustrating the next step in an exemplary aspect outlined in FIG. 9 wherein the FFPE tissue is then contacted with an array of spatially-barcoded oligonucleotides probes. The released oligonucleotide probes can diffuse into the tissue and hybridize to DNA probes that were first hybridized to RNAs in a tissue section and ligated together.

FIG. 11 is a schematic illustrating a step in the exemplary aspect shown in FIG. 10, where the spatially-barcoded oligonucleotides are extended with a DNA polymerase to form a complement to the DNA probes hybridized to tissue RNAs. These extension products can then be subject to PCR to form a double-stranded spatially-barcoded library of the ligated RNA hybridization probes.

FIG. 12 shows two examples of Agilent Bioanalyzer traces (DNA 1000 kit) of two cDNA libraries synthesized by transcription in situ on an FFPE tissue section followed by PCR on the ground up FFPE tissue containing newly synthesized first-strand cDNA.

FIG. 13 shows the size distribution of cDNA inserts from a spatially barcoded sequencing library constructed by the method outlined in FIGS. 2 and 3. The sizes were determined by paired-end DNA sequencing of the inserts.

FIG. 14 shows the abundance distribution of the RNA isoforms sequenced in the cDNA library described in FIG. 13. A little over 3,000 different transcript isoforms were identified, with a wide range of abundances when expressed in transcripts per million (TPM).

FIG. 15 is a plot of the abundance of the 244,000 different spatial barcodes from the sequencing library described in FIG. 13. The number of reads per barcode (x axis) is plotted against the number of different barcodes that have that read count. About 42,000 of the 244,000 possible barcodes gave at least 10 reads per barcode.

FIG. 16 shows a spatial plot of barcode locations from the sequencing library described in FIG. 13-15. The location of any barcode with less than 20 instances in the library is plotted as white, while the location of barcodes with 20-60 reads is plotted as a grey gradient, and the position of any barcode with over 60 reads is shown as a dark grey spot. It is clear in the figure that most barcodes are located in the area where the FFPE tissue was fixed to the tissue slide.

FIG. 17 shows a spatial plot of ErbB2 and Mdm2 mRNA expression in the same zoomed-in area of the tissue section assayed in FIG. 16. It can clearly be seen that the spatial expression pattern of the two genes are different.

### DETAILED DESCRIPTION OF THE INVENTION

Unless defined otherwise, all technical and scientific terms used herein have the same meaning as commonly understood by one of ordinary skill in the art to which the disclosure belongs. The cited references are incorporated by reference in their entirety, or in part wherein the parts of the references relevant to the purpose of their citation are incorporated.

When introducing elements of the present disclosure or the various versions, aspect(s) or aspects thereof, the articles “a”, “an”, “the” and “said” are intended to mean that there are one or more of the elements. The terms “comprising”, “including” and “having” are intended to be inclusive and mean that there can be additional elements other than the listed elements.

In an embodiment, there is disclosed a method for detecting nucleic acids, the method comprising: providing a tissue sample; providing an array comprising a plurality of oligonucleotide probes attached to a surface of the array, wherein each oligonucleotide probe, of the plurality of oligonucleotide probes, comprises a location barcode sequence, a primer binding sequence, and a priming sequence; releasing the plurality of oligonucleotide probes from the array surface; contacting the tissue sample with the released oligonucleotide probes; and allowing the released oligonucleotide probes to diffuse into the tissue sample.

In an embodiment, there is disclosed a method for detecting nucleic acids, the method comprising: providing a tissue sample; providing a microarray that comprises a plurality of oligonucleotide probes attached to a microarray surface, wherein the oligonucleotide probes comprise a location barcode, a primer binding sequence, and a priming sequence; releasing the plurality of oligonucleotide probes from the microarray surface while substantially maintaining their locations on the microarray surface; contacting the tissue sample with the oligonucleotide probes; allowing the oligonucleotide probes to diffuse into the tissue sample, and incubating the oligonucleotide probes and the tissue sample for a sufficient time to allow the plurality of oligonucleotide probes to hybridize to target nucleic acids within the tissue sample; extending the priming sequence on the oligonucleotide probes to produce a primer extension product comprising the location barcode; amplifying the primer extension product to result in amplified products, and sequencing the amplified products.

In an embodiment, the target nucleic acids comprise mRNAs, and the priming sequence comprises oligo(dT).

In an embodiment, the target nucleic acids comprise cDNAs which each comprise at least a first-strand cDNA.

In an embodiment, the priming sequence binds to a sequence in the first strand cDNA.

In an embodiment, wherein the target nucleic acids comprise cDNAs synthesized in the presence of a template switching oligonucleotide, and the priming sequence binds to a sequence added by the template switching oligonucleotide.

In an embodiment, the first strand cDNA comprises an adapter ligated to its 3′-end, and the priming sequence binds to the adapter.

In an embodiment, said plurality of oligonucleotide probes are attached to the microarray surface by hybridization.

In an embodiment, a plurality of oligonucleotide probes sharing the same location barcode are bound by a microarray feature comprising a complementary sequence to that location barcode.

In an embodiment, the plurality of oligonucleotide probes are attached to the microarray surface covalently.

In an embodiment, said plurality of probes are released from the microarray surface by cleavage with gaseous ammonia.

In an embodiment, said plurality of probes are released from the microarray surface by photocleavage.

In an embodiment, said plurality of probes are released from the microarray surface by a restriction enzyme.

In an embodiment, said plurality of probes are released from the microarray surface by denaturation.

In an embodiment, the tissue sample is contacted with the oligonucleotide probes after the oligonucleotide probes are released from the microarray surface.

In an embodiment, the tissue sample is contacted with the oligonucleotide probes before the oligonucleotide probes are released from the microarray surface.

In an embodiment, the target nucleic acids comprise nucleic acid tags indicative of particular antibodies.

The term “genome,” as used herein, refers to all nucleic acid sequences (coding and non-coding) and elements present in any virus, single cell (prokaryote or eukaryote) or each cell type in a metazoan organism. The term genome also applies to any naturally occurring or induced variation of these sequences that can be present in a mutant or disease variant of any virus, cell, or cell type. Genomic sequences include, but are not limited to, those involved in the maintenance, replication, segregation, and generation of higher order structures (e.g. folding and compaction of DNA in chromatin and chromosomes), or other functions, as well as all of the coding regions and their corresponding regulatory elements needed to produce and maintain each virus, cell, or cell type in a given organism.

The term “nucleotide” is intended to include those moieties that contain not only the known purine and pyrimidine bases, but also other heterocyclic bases that have been modified. Such modifications include methylated purines or pyrimidines, acylated purines or pyrimidines, alkylated riboses or other heterocycles. In addition, the term “nucleotide” includes those moieties that contain hapten or fluorescent labels and can contain not only conventional ribose and deoxyribose sugars, but other sugars as well. Modified nucleosides or nucleotides also include modifications on the sugar moiety, e.g., wherein one or more of the hydroxyl groups are replaced with halogen atoms or aliphatic groups, are functionalized as ethers, amines, or the likes.

The term “nucleic acid” and “polynucleotide” are used interchangeably herein to describe a polymer of any length, e.g., greater than about 2 bases, greater than about 10 bases, greater than about 100 bases, greater than about 500 bases, greater than 1000 bases, up to about 10,000 or more bases composed of nucleotides, e.g., deoxyribonucleotides or ribonucleotides, and can be produced enzymatically or synthetically (e.g., PNA as described in U.S. Pat. No. 5,948,902 and the references cited therein) which can hybridize with naturally occurring nucleic acids in a sequence specific manner analogous to that of two naturally occurring nucleic acids, e.g., can participate in Watson-Crick base pairing interactions. Polynucleotides can have any three-dimensional structure, and can perform any function, known or unknown. The following are non-limiting examples of polynucleotides: coding or non-coding regions of a gene or gene fragment, loci (locus) defined from linkage analysis, exons, introns, messenger RNA (mRNA), transfer RNA, ribosomal RNA, ribozymes, cDNA, recombinant polynucleotides, branched polynucleotides, plasmids, vectors, isolated DNA of any sequence, isolated RNA of any sequence, nucleic acid probes, and primers. A polynucleotide can comprise modified nucleotides, such as methylated nucleotides and nucleotide analogs, or xenonucleic acids (XNAs.) If present, modifications to the nucleotide structure can be imparted before or after assembly of the polymer. The sequence of nucleotides can be interrupted by non-nucleotide components. A polynucleotide can be further modified after polymerization, such as by conjugation with a labeling component. Naturally-occurring nucleotides include guanine, cytosine, adenine, thymine, uracil (G, C, A, T and U respectively). DNA and RNA have a deoxyribose and ribose sugar backbone, respectively, whereas PNA's backbone is composed of repeating N-(2-aminoethyl)-glycine units linked by peptide bonds. In PNA various purine and pyrimidine bases are linked to the backbone by methylene carbonyl bonds. A locked nucleic acid (LNA), often referred to as inaccessible RNA, is a modified RNA nucleotide. The ribose moiety of an LNA nucleotide is modified with an extra bridge connecting the 2′ oxygen and 4′ carbon. The bridge “locks” the ribose in the 3′-endo (North) conformation, which is often found in the A-form duplexes. LNA nucleotides can be mixed with DNA or RNA residues in the oligonucleotide whenever desired. The term “unstructured nucleic acid”, or “UNA”, is a nucleic acid containing non-natural nucleotides that bind to each other with reduced stability. For example, an unstructured nucleic acid can contain a G′ residue and a C′ residue, where these residues correspond to non-naturally occurring forms, i.e., analogs, of G and C that base pair with each other with reduced stability, but retain an ability to base pair with naturally occurring C and G residues, respectively. Unstructured nucleic acid is described in US Patent Application 20050233340, which is incorporated by reference herein for disclosure of UNA.

The term “oligonucleotide” as used herein denotes a multimer of nucleotide of from about 2 to 500 nucleotides in length. Oligonucleotides can be synthetic or can be made enzymatically, and, in some aspects, are 30 to 150 nucleotides in length. Oligonucleotides can contain ribonucleotide monomers (i.e., can be oligoribonucleotides) or deoxyribonucleotide monomers, or both ribonucleotide monomers and deoxyribonucleotide monomers. An oligonucleotide can be 10 to 20, 11 to 30, 31 to 40, 41 to 50, 51-60, 61 to 70, 71 to 80, 80 to 100, 100 to 150 or 150 to 200 nucleotides in length, for example.

As used herein, a “target nucleic acid” refers to a nucleic acid comprising a sequence whose quantity or degree of representation (e.g., copy number) or sequence identity is being assayed. A sample will typically contain one or more target nucleic acids. Target nucleic acids can comprise either RNA, DNA, or both. The RNA can be mRNA, tRNA, rRNA, viral RNA, small nuclear RNA (snRNA), small nucleolar RNA (snoRNA), microRNA (miRNA), small interfering RNA (siRNA), piwi-interacting RNA (piRNA), ribozymal RNA, antisense RNA or non-coding RNA. Specifically, the target nucleic acids can include RNAs that are not polyadenylated. In addition, target nucleic acids can comprise nucleic acids that either occur naturally in a cell, nucleic acids that are introduced into living cells (e.g., by transfection with plasmids, biolistic introduction, or viral infection), or nucleic acids that are introduced into cells or samples after fixation but prior to analysis.

The term “primer” refers to an oligonucleotide capable of acting as a point of initiation of synthesis along a complementary strand when conditions are suitable for synthesis of a primer extension product. The synthesizing conditions for DNA include the presence of at least one deoxyribonucleotide triphosphate, and typically four different deoxyribonucleotide triphosphates, and at least one polymerization-inducing agent such as reverse transcriptase or DNA polymerase. These are present in a suitable buffer, which can include constituents which are co-factors or which affect conditions such as pH and the like at various suitable temperatures. A primer is preferably a single stranded sequence, such that amplification efficiency is optimized, but double stranded sequences can be utilized.

The term “probe” or “oligonucleotide probe” refers to an oligonucleotide or a set of oligonucleotides that hybridizes to a target sequence. In some aspects, a probe includes about eight nucleotides, about 10 nucleotides, about 15 nucleotides, about 20 nucleotides, about 25 nucleotides, about 30 nucleotides, about 40 nucleotides, about 50 nucleotides, about 60 nucleotides, about 70 nucleotides, about 75 nucleotides, about 80 nucleotides, about 90 nucleotides, about 100 nucleotides, about 110 nucleotides, about 115 nucleotides, about 120 nucleotides, about 130 nucleotides, about 140 nucleotides, about 150 nucleotides, about 175 nucleotides, about 187 nucleotides, about 200 nucleotides, about 225 nucleotides, and about 250 nucleotides. A probe can further include a detectable label. Detectable labels include, but are not limited to, a fluorophore (e.g., Texas-Red®, Fluorescein isothiocyanate, etc.), radioactive labels, mass tag labels, and haptens, (e.g., biotin). Preferred detectable labels comprise atoms, molecules, or complexes which are not normally present at a high concentration in the relevant areas of the sample. A detectable label can be covalently attached directly to a probe oligonucleotide, e.g., located at the probe's 5′ end or at the probe's 3′ end. A probe including a fluorophore can also further include a quencher, e.g., Black Hole Quencher™, Iowa Black™ etc. In some aspects, the probes do not contain a detectable label.

Disclosed herein are methods for detecting nucleic acids and their locations. In some aspects, oligonucleotide probes that contain primers and location-specific barcodes can be arranged in a location-specific manner on an array surface, but are not covalently attached to the array surface. The oligonucleotides can be allowed to diffuse into a target tissue, hybridize to target nucleic acids for primer extension, and the extension products (or amplified products thereof) can be sequenced. The location barcodes in the extension products can be indicative of where the target nucleic acids are located in the tissue. The methods perform many of the biochemical steps in situ, and do not require diffusion of the target nucleic acids to the array surface. In addition, the use of pre-released oligonucleotide probes on the array can enable the location-encoded oligonucleotide probes to diffuse into the tissue, interacting directly with the target nucleic acids there. In different aspects, the oligonucleotide probes can include additional sequence elements. For example, the oligonucleotide probes, which can contain location barcode sequences, can be cleaved and used as primers for cDNA synthesis, or as template switching oligos, or as primer extension oligos. The methods advantageously allow for cDNA synthesis and subsequent amplification (e.g., PCR) to be performed without releasing the RNA from the tissue section or purifying the in situ synthesized cDNA from the tissue sample. The methods also advantageously allow for determining the strandedness of the RNA sequences.

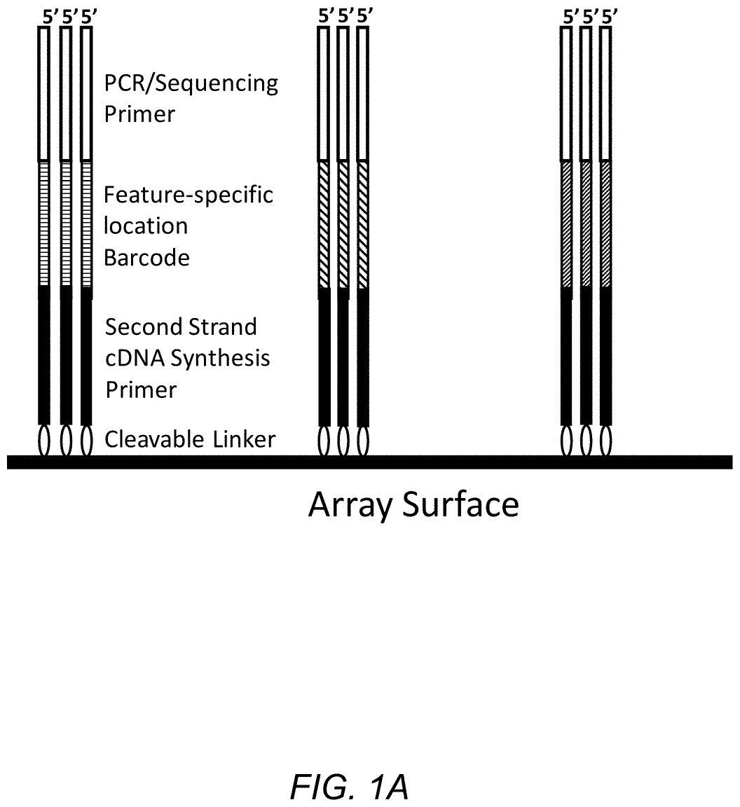

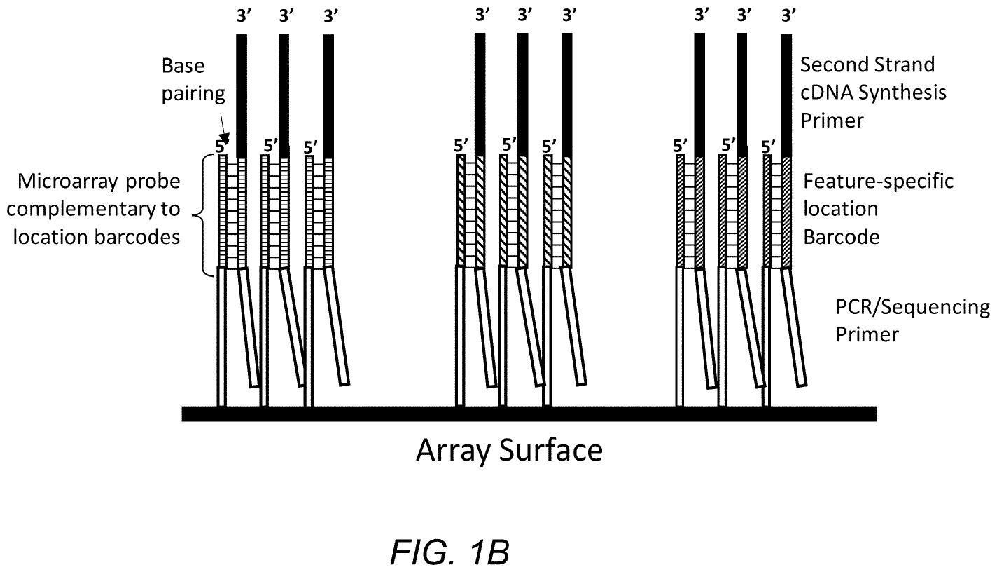

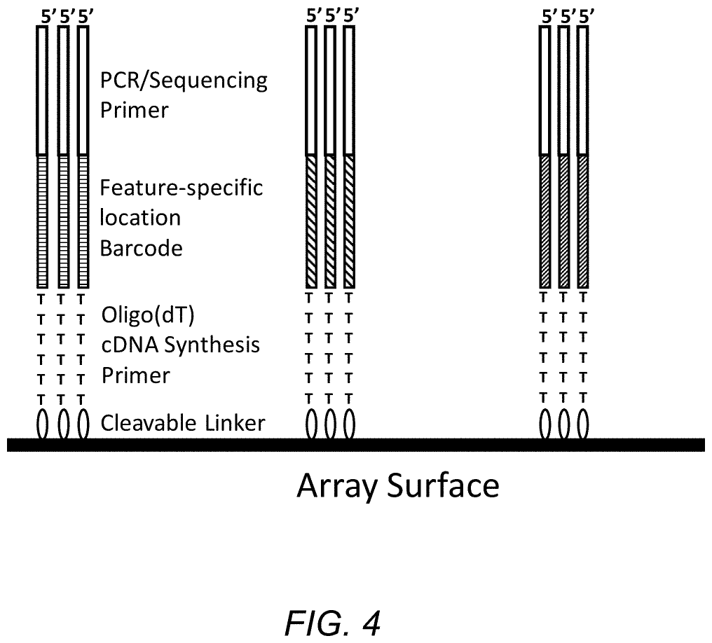

A plurality of non-random, defined oligonucleotide probes (also referred to herein as “first strand cDNA primers”, “oligos”, and “probes”) can be generated on a surface of an array (also interchangeably called “microarray” in this application). These oligonucleotide probes can be attached to an array surface covalently, or non-covalently, such as by hybridization (FIG. 1B). In some aspects, an oligonucleotide probe can comprise at least two different subsequences wherein each of the two different subsequences can bind to a different site in a target nucleic acid present in a tissue. In some aspects, an oligonucleotide probe can comprise both known and randomized, degenerate, or unknown sequences. Methods for generating degenerate or randomized sequences are known in the art. The oligonucleotide probes can have the following sequences (listed 5′ to 3′): a PCR primer sequence, a feature-specific barcode sequence (also referred to herein as a “location barcode”, which tells the location of the oligonucleotide probe on the array surface), a primer sequence for querying the sequences from a tissue (e.g., an oligo-dT (FIG. 4), a second-strand cDNA synthesis primer (FIG. 1A), or a gene-specific sequence), and optionally a cleavable linker at the 3′ end. Additionally, the oligonucleotide probes can contain a molecular barcode, a sample index, and/or other sequences.

As shown in FIG. 1A, an array of oligonucleotide probes can be attached to the array surface with a cleavable linker. As shown in FIG. 1B, an array of oligonucleotide probes can hybridize to a first oligonucleotide (also referred to herein as an “array feature”) which includes a complementary location barcode to the oligonucleotide probe.

As used herein, a “location barcode sequence” refers to a known nucleotide sequence that is used to identify the oligonucleotide probe location on the array surface. Different locations on the array surface can correspond to different regions of a tissue, and can be distinguished by their different location barcode sequences.

Each separate location in the array can include a plurality of oligonucleotide probes, such as one or more oligonucleotide probes, or two or more oligonucleotide probes. As shown in FIG. 1A, the plurality of oligonucleotide probes in a single location can include the same feature-specific location barcode. Each location of the array can include different feature-specific location barcodes.

As shown in FIG. 1B, more than one type of oligonucleotide can share a location barcode, so that several types of oligonucleotide probe can be present on a first oligonucleotide with a complementary location barcode. For example, one array feature with a location barcode can capture oligonucleotide probes with an oligo(dT) sequence at the 3′ end, together with oligonucleotides with a random priming sequence at the 3′ end, as well as oligonucleotide probes with a specific priming sequence at the 3′ end; as long as each of these types of oligonucleotide probes has the complementary location barcode for that array feature.

Each of these oligonucleotide probes can bind to a different target nucleic acid in the tissue, while enabling transfer of the same location barcode to each of the target nucleic acids. Depending on the desired length and melting temperature of the location barcode sequences, the array probes could be designed to include complementary sequences to other regions of the probe library, such as the primer site adjacent to the location barcode.

In this manner, there is an array including a plurality of oligonucleotide probes present in each location of the array, in which each oligonucleotide probe includes a location barcode unique to the location. The method includes separating the oligonucleotide probes from the array surface in a manner so that the oligonucleotide probes can remain in their unique location. If the method utilizes an oligonucleotide probe as illustrated in FIG. 1A, then the method can include cleaving the linker. If the method utilizes an oligonucleotide probe as illustrated in FIG. 1B, then the method can include separating the hybridized sequences. It should be appreciated that the particular method of attaching the oligonucleotide probes to the array surface and subsequently separating the oligonucleotide probes from the array surface can vary so long as the oligonucleotide probes remain in their respective locations, are free from the array surface, and are able to diffuse into the tissue.

Referring to FIG. 1A, oligonucleotide probes can comprise at least one, two, three, four, or more, cleavage sites. Oligonucleotides can be cleaved from the array surface at specific cleavage sites by light, heat, a chemical, or enzymes such as RNAses or restriction enzymes. Cleavage chemicals can be applied to the array in liquid or gaseous form. Such cleavage can result in oligonucleotides of varying lengths, including, but not limited to, any length from 15 to 250 base pairs (bp), 18 bp, 25 bp, 30 bp, 35 bp, 40 bp, 50 bp, 60 bp, 70 bp, 75 bp, 80 bp, 90 bp, 100 bp, 110 bp, 115 bp, 120 bp, 125 bp, 130 bp, 140 bp, 150 bp, 175 bp, 200 bp, 225 bp, and/or 250 bp.

Oligonucleotide probes can be cleaved on the array surface and left in place, maintaining spatial positioning, in the absence of a covalent linkage between the array and the oligonucleotide probe. Gas phase deprotection reagents (e.g. gaseous ammonia or methylamine) can be used to cleave oligonucleotide probes from the array surface. For example, ester linkers can be cleaved by gas phase amines, but the lack of aqueous solvents can prevent the oligonucleotide probes from migrating away from their spatial positioning on the array surface. As an example of cleavage, we have previously found (described in U.S. Pat. No. 9,834,814 and references therein) that cleavage can be performed using gaseous ammonia so that the array probe oligos, once cleaved, stay in the same position on the array slide as long as the slide stays dry. Deprotection side products can be removed by washing the array with a solvent or a solvent mixture in which the oligonucleotide probes are not appreciably soluble. Non-limiting examples of such solvents include acetonitrile and toluene. In this manner, the oligonucleotide probes can maintain their spatial positioning.

In some aspects, more than one cleavable linker or mode of attachment can be used to initially attach the oligonucleotide probes to the array. For example, an oligonucleotide probe synthesized on the array can contain 2, 3, 4, or more cleavable linkers, such that the oligonucleotide probe can be cleaved into 3, 4, 5, or more shorter oligonucleotides by the cleavage treatment. This aspect enables oligonucleotides synthesized in one array feature to participate in amplification or primer extension assays on more than one specific target nucleic acid in the tissue. For example, one 100 mer oligonucleotide probe can be cleaved into four 25 mer primers that are two pairs of primers, which can be used to amplify two specific targets by PCR. Also, more than one type of cleavable linker or mode of attachment can be used. In this way, different sets of oligonucleotide probes can be released at different times. For example, treatment with gaseous ammonia can cleave one type of linker, while a second type of linker can be photocleavable.

Referring to FIG. 1B, the oligonucleotide probes can be captured on an array by hybridization to array features including complementary sequences to the location barcodes, and other parts of the probe, if desired. The oligonucleotide probes of FIG. 1B can be absent a cleavable linker and can be oriented with the 3′ end away from the array surface. In particular, a first oligonucleotide is attached to the array surface, and can include at its 5′ end a sequence that is complementary to the feature-specific location barcode of the oligonucleotide probe. The oligonucleotide probe can hybridize to the 5′ end of the first oligonucleotide, effectively attaching the oligonucleotide probe to the array surface, but with the 3′ end of the oligonucleotide probe facing away from the array surface.

The first oligonucleotide can vary in length so long as a portion near its 5′ end can hybridize with the feature-specific location barcode of the oligonucleotide probe. The use of the first oligonucleotide allows attachment of the oligonucleotide probe to the array surface, but without attaching the oligonucleotide probe directly to the array surface, and/or with the 3′ end of the oligonucleotide probe facing “up” or away from the array surface.

In some aspects, where the oligonucleotide probes are hybridized rather than covalently linked to the array surface, the probes are recruited or “sorted” to the desired locations on the array surface. Thus, a mixture of oligonucleotide probes in solution can hybridize to an array with covalently bound first oligonucleotides (“index oligos”) that are unique in each location and at least partially complementary to some of the probes in the mixture. In some aspects, the soluble oligonucleotide probes can comprise location barcodes, and can hybridize to a first oligonucleotide which can comprise a sequence complementary to the location barcode of the oligonucleotide probe.

These hybridized oligonucleotides could be removed by denaturing conditions such as high pH, addition of formamide, or a temperature above the Tm of the duplex. In some aspects, the hybridized oligonucleotides can contain cleavable sites such as a restriction enzyme recognition site, a deoxyuridine residue, or one or more RNA nucleotides, such that these oligonucleotides can be cleaved by an enzyme such as a restriction enzyme, a mixture of Uracil DNA glycosylase (UDG) and the DNA glycosylase-lyase Endonuclease VIII, or an RNAse such as RNAseH. In some aspects, the array oligos could comprise recognition sites such that they be cleaved by a nicking endonuclease, releasing the hybridized oligonucleotide probe sequence into the tissue. Alternatively, the covalently bound oligonucleotide probes could be removed by cleavage conditions, either before or after dissociation of the hybridized oligonucleotides.

Releasing the oligonucleotide probes from the array surface, while substantially maintaining their locations on the array surface, can be done in any way known in the art. With careful design of the oligonucleotides, linkers, and conditions, it will be possible to allow a variety of sizes of oligonucleotide probes to be removed from the array surface in different conditions. The method includes allowing the detached oligonucleotide probes to diffuse into a tissue. By “diffuse into the tissue”, it is understood that the oligonucleotide probe including the feature-specific location barcode can is free to move, spread out, and/or enter into a mass of the tissue, as opposed to remaining on a surface of or an interface with the tissue. [Please confirm this definition.] The method can be performed on a solid tissue sample, e.g., a tissue section from a formalin-fixed paraffin-embedded (FFPE) tissue. The solid tissue can be the product of a biopsy, e.g., a tumor biopsy. Alternatively, the method can be performed with a fresh or fresh frozen tissue section.

The term “sample” as used herein refers to an object containing nucleic acid molecules. The consistency of the sample is typically in such a way that the nucleic acid molecules of interest have an inhomogeneous or unequal distribution. Preferably, the nucleic acids should not be in solution. Preferred samples are non-fluidic, gel-like, fixated or solid. Examples of suitable samples are tissue sections, tissue blocks, a gel layer, a cell, a cell layer, a tissue array, yeasts or bacteria on a culture plate, membrane, paper or fabric, or a carrier with spots of isolated or synthetic nucleic acid molecules. In general, the sample can comprise a carrier made of glass, plastic, paper, a membrane (e.g. nitrocellulose) or fabric. For example, a tissue section is usually applied on a glass slide or coverslip. A cell layer could also be provided on a glass slide or on a plastic dish. Unicellular organisms can be provided on culture plates, on filter paper or on a fabric. The nucleic acid molecule can be within the sample for example within a fixed cell, within a gel or within a tissue. Alternatively, the nucleic acid molecules can be provided on the surface of a sample like a array (2D array on a solid substrate; usually a glass slide or silicon thin film cell), for example a DNA array also commonly known as DNA chip or biochip.

In an aspect, the sample is a tissue section. The tissue section and also other samples (e.g. cells or unicellular organisms) can be frozen (fresh frozen or fixed frozen), fixed (formaldehyde fixed, formalin fixed, methanol fixed, ethanol fixed, acetone fixed or glutaraldehyde fixed) and/or embedded (using paraffin, Epon or other plastic resin). Such tissue sections can be prepared with a standard steel microtome blade or glass and diamond knives as routinely used for electron microscopic sections. Furthermore, small blocks of tissue (less than 15 mm thick) can be processed as whole mounts. If the nucleic acid molecules are on the surface of the sample, thickness of the sample does not really matter so that any thickness could be used. If the nucleic acid molecules are located within the sample like tissue slides, thickness should be in a range that the nucleic acid molecules could move out of the sample to the target surface. A thickness of such samples can be, for example, 1 micrometer to 1 mm and, for example, from 2 micrometers to 10 micrometers.

Disclosed herein, inter alia, are methods for performing spatial RNA-sequencing, which means determining the sequence and location of RNAs in a tissue section using an array of released oligonucleotide probes. Although the details of the different aspects vary, the aspects generally describe methods of combining location-specific barcode information imparted from the array feature, to append location-specific barcode information and amplification sequences to sequence information from the tissue sample.

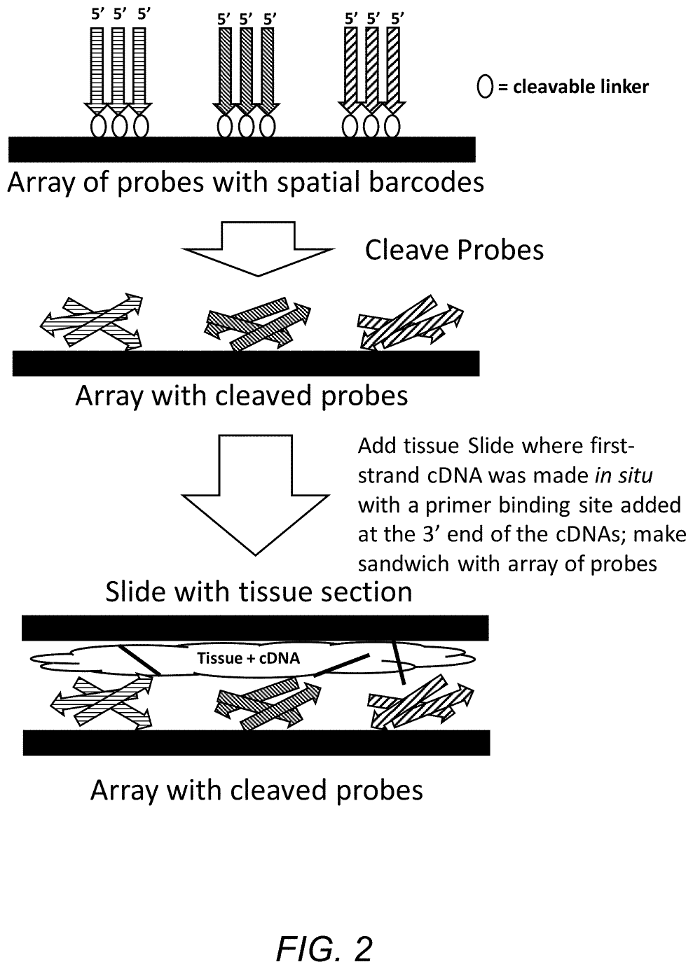

FIG. 2 illustrates an overview of a method of detecting nucleic acids. Oligonucleotide probes can be provided on an array surface as discussed above with regard to FIGS. 1A and 1B. The oligonucleotide probes can be released in place, such as cleaved as shown in FIG. 2. It is noted that although FIG. 2 illustrates the oligonucleotide probes of FIG. 1A, the steps of the method shown in FIG. 2 are equally applicable with the oligonucleotide probes of FIG. 1B, or FIG. 4. The array including the released oligonucleotide probes can be placed in contact with a tissue slide containing the tissue to form a “sandwich”. When the sandwich is made between the tissue and the array under aqueous conditions, the released oligonucleotide probes comprising location barcodes can be allowed to diffuse into the tissue present on the tissue slide. The oligonucleotide probes can hybridize with target nucleic acids that are in the tissue, and can facilitate primer extension and/or amplification of the target nucleic acids with the location barcodes incorporated in the extension/amplification products. Upon sequencing of such extension/amplification products, the identity and location of the target nucleic acids can be determined based on the sequence and location barcode, respectively. In some aspects, the array or the tissue can be embedded in a gel-like matrix instead of just in buffer.

The array including the released oligonucleotides has been discussed above. With regard to the tissue slide, suitable tissue samples can include FFPE tissue sections and fresh or frozen tissue sections. If an FFPE tissue section is used, the section can be de-paraffinized, using xylene or other standard treatments. The FFPE tissue can also be pepsin-treated before use if desired, which in some instances can increase access to RNA or other target molecules. In some aspects, the RNA in the tissue can be partially fragmented by sonication, or enzymatic or chemical treatment, in order to make the RNA more accessible to enzymes or primers. Alternatively, or in parallel, if one wants to preserve the protein structure of the tissue, then treatment in an antigen-retrieval or similar buffer can be performed. At some point in the method, the tissue can be stained and a microscopic image captured. Alternatively, spatial RNA sequence information can be obtained by one section of FFPE tissue, while imaging, FISH, or immunohistochemistry can be performed on an adjacent section, and the resulting data from the adjacent sections could be combined. Ultimately, deeper biological insights can be obtained by combining several data types, including image data, sequence data (from RNA, or from surrogate sequences representing other biological markers), protein or antibody binding data, etc.

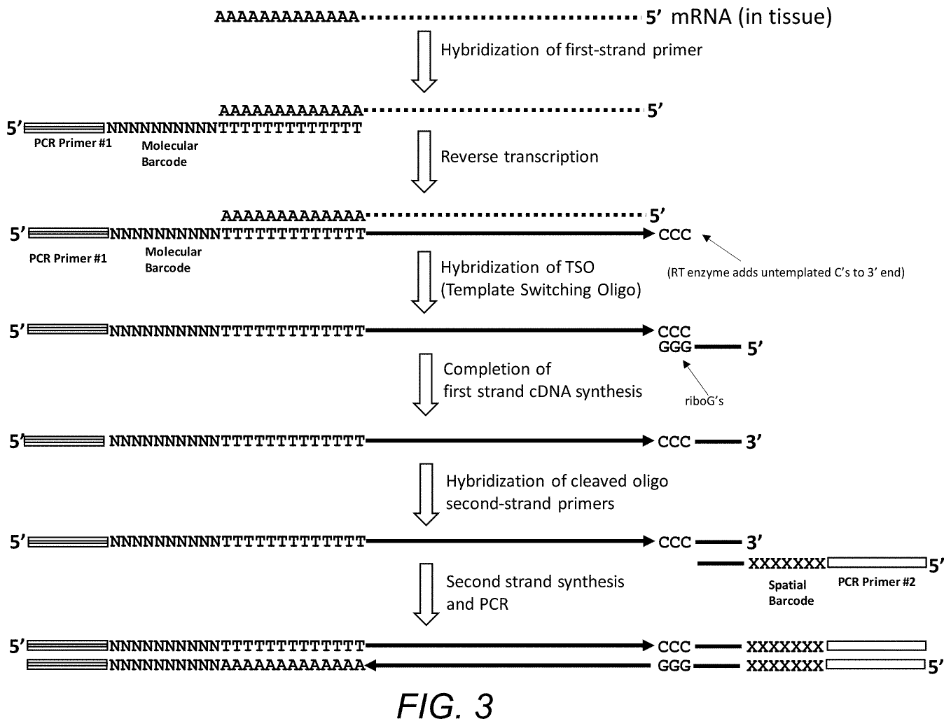

The target nucleic acid can be, for example, mRNA, cDNA, or nucleic acid tags used to label particular antibodies. The target nucleic acids in the tissue can be, for example, mRNA, cDNA, or other oligonucleotides such as barcode oligonucleotides attached to specific proteins or antibodies. In some aspects, the target is cDNA, which can be synthesized in the (entire) tissue, prior to exposing the tissue to the arrayed oligonucleotides. Thus, before the array slide and the tissue slide are placed together to form the “sandwich”, the RNA in the tissue section is reverse transcribed to form a first strand cDNA (e.g., see FIG. 3).

In FIG. 3, there is illustrated a further detailed process of the method of detecting nucleic acid. The first line illustrates a tissue on a tissue slide, in which the tissue includes target nucleic acids in the form of mRNA. A solution can be added to the tissue section. The can contain an oligo(dT) primer, reverse transcriptase and its buffer, dNTPs, and a template-switching oligo (TSO) (BioTechniques 30, no. 4 (2001): 892-897). The oligo(dT) primer can have a PCR primer binding site at its 5′ end and an oligo d(T) region at its 3′ end. A molecular barcode sequence, described above, can be inserted, for example between these two regions if desired (FIG. 3, line 2). The solution can be gently spread across the tissue section, and the tissue slide can be incubated under low temperature conditions allowing the oligo(dT) primer to hybridize to a poly(A) tail on mRNAs in the tissue section. The temperature can then be raised to allow the reverse transcriptase to extend from the oligo-dT tail of the oligonucleotide probe sequence. When the reverse transcriptase reaches the end of the mRNA fragment, it tends to add 3 untemplated C residues to the 3′ end of the first strand cDNA (FIG. 3, line 3).

These three C's can hybridize to three ribo-G residues at the 3′ end of the TSO (FIG. 3, line 4). The TSO includes three ribo-G residues at the 3′ end and a second strand cDNA priming region upstream. When the TSO hybridizes to the untemplated Cs at the end of the first strand cDNA, it can serve as a template to further extend the first stand of the cDNA. The term “template switching oligo,” or “TSO,” refers to an oligonucleotide that can hybridize to the end of a nascent first-strand cDNA chain created by reverse transcriptase, enabling continuation of cDNA synthesis. In some aspects, the nascent chain has 3 or more cytosine residues at the 3′ end. In these aspects, the TSO comprises 3 riboguanosine residues at the 3′ end, downstream from a primer or adapter sequence. In some aspects, the TSO can comprise a spatial barcode, sample index, or molecular barcode sequence (e.g., an oligonucleotide library) in addition to the primer or adapter sequence.

As used herein, a “molecular barcode sequence” refers to a nucleotide sequence that can be used to differentiate nucleic acids arising from different template molecules. Molecular barcode sequences can be used to identify duplicate molecules arising from the same template, and/or can be used to correct for errors arising during PCR amplification or sequencing. In some aspects, the molecular barcode sequences can be composed of random nucleotides, or a mixture of random and known nucleotides. A molecular barcode sequence can be at the 5′-end, the 3′-end or in the middle of an oligonucleotide.

Barcode sequences, such as location barcode sequences and molecular barcode sequences, can vary widely in size and composition; the following references provide guidance for selecting sets of barcode sequences appropriate for particular aspects: Brenner, U.S. Pat. No. 5,635,400; Brenner et al, Proc. Natl. Acad. Sci., 97: 1665-1670 (2000); Shoemaker et al, Nature Genetics, 14: 450-456 (1996); Morris et al, European patent publication 0799897A1; Wallace, U.S. Pat. No. 5,981,179; and the like. In particular aspects, a barcode sequence can have a length in range of from 4 to 36 nucleotides, or from 6 to 30 nucleotides, or from 8 to 20 nucleotides. Typically, the barcode sequence can range from about 5 nucleotides to about 20 nucleotides.

At the end of the first strand cDNA synthesis (FIG. 3, line 5), the tissue slide includes tissue with a first strand cDNA, which is brought into contact a DNA polymerase, buffer, dNTPs, and the array of released oligonucleotide probes to form a sandwich (FIG. 3, line 6). The sandwich is incubated under conditions to allow the DNA polymerase to synthesize the second strand cDNA using the released, location-barcoded oligonucleotides as primers (FIG. 3, line 7). The first strand is extended to include the location barcode and the PCR primer region at its 3′ end and retaining the molecular barcode and PCR primer region at its 5′ end. The complementary second strand also includes a PCR primer region and the molecular barcode at its 3′ end and the location barcode and a PCR primer region at its 3′ end.

After this the tissue slide can be removed from the released oligonucleotide array slide, the tissue and solution can be scraped into a tube, and PCR can be performed using PCR primers complementary to the priming sequences at the 5′ and 3′ ends of the cDNA, which were put into place by the oligo(dT) primer and the TSO. This library of cDNA PCR products can then be sequenced.

The location on the array of the first strand cDNA primers can then be deconvoluted by examination of the location barcodes. Subsequently, the locations of the mRNA sequences determined by the location barcodes can be aligned with the image of the tissue section obtained prior to the in situ RNA-sequencing. In some aspects, the tissue section can have a characteristic shape, size, or dimensions, which helps to determine whether a signal is noise or not, since a signal outside of the section should be noise. In this way, the image of the tissue section can be aligned with the mRNA sequences obtained from the in situ RNA sequencing. Location barcodes corresponding to regions of the array that were not in contact with the tissue section will not be represented in the RNA sequencing library.

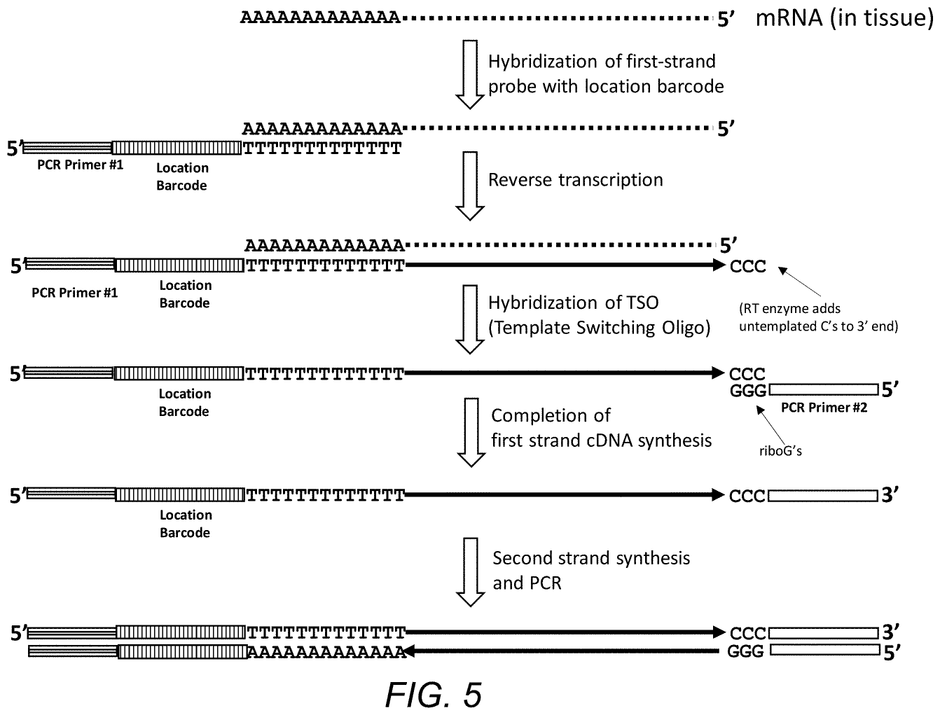

Another aspect of the invention, is a method utilizing the oligonucleotide probes of FIG. 4. As shown in FIG. 4, the oligonucleotide probe can include at its 5′ end a PCR primer sequence, a location barcode, an oligo(dT) cDNA synthesis primer sequence, and optionally a cleavable linker at its 3′ end. The oligonucleotide probe of FIG. 1B can be used in a method after first strand cDNA synthesis has been completed, as shown in FIG. 3. The oligonucleotide probe of FIG. 4 can be used in a method before first strand cDNA synthesis has been completed, as shown in FIG. 5.

The tissue slide including the target nucleic acid can be brought into contact with reverse transcriptase and its buffer, dNTPs, and a TSO (FIG. 5, line 1). The released oligonucleotide probes including the location barcode, and oligo(dT) cDNA synthesis primer sequence from FIG. 4 can be applied to the tissue slide to form the sandwich. The released oligonucleotide probes can then diffuse into the tissue, allowing the oligo-dT on the 3′ end of the oligonucleotide probes to hybridize to the poly(A) tail of mRNAs found in the tissue section (FIG. 5, line 2). This can be aided by placing the sandwich briefly at a low temperature to facilitate annealing. The temperature can then be raised to allow the reverse transcriptase to extend from the oligo-dT tail of the oligonucleotide probe (FIG. 5, line 3). When the reverse transcriptase reaches the end of the mRNA fragment, the TSO hybridizes to the untemplated C residues allowing it to be copied by the reverse transcriptase (FIG. 5, line 4). In this aspect, the TSO includes three regions (listed from 5′ to 3′): a PCR primer sequence, a molecular barcode sequence (ranging from about 5 to 20 nucleotides—not shown), and three ribo-G residues. After first strand cDNA synthesis, the first strand includes a location barcode near the 5′ end and a molecular barcode (not shown) near the 3′ end (FIG. 5, line 5). The tissue slide can be removed from the array slide and PCR is performed using PCR primers complementary to the PCR priming sequences at the 5′ and 3′ ends of the first strand cDNA (FIG. 5, line 6). This library of cDNA PCR products can then be sequenced. Alternatively, instead of using a TSO, the 3′ end of the first strand cDNA could be ligated to a single-stranded adapter sequence containing a molecular barcode (if desired) and a PCR primer sequence. This could be done after isolating the first-strand cDNA from the tissue section if desired. After ligation, PCR can be performed to amplify the cDNAs and the products are analyzed by DNA sequencing.

In another exemplary aspect using released oligonucleotide arrays, TSOs with location barcodes are printed on the array surface. This allows the user to perform most of the first-strand cDNA synthesis with a non-arrayed oligo(dT) primer in the tissue before making the “sandwich” between the array slide and tissue sample slide. Making the sandwich allows the TSOs to diffuse into the tissue from the surface of the array, to hybridize to the 3′ non-templated CCC residues of the first strand cDNA in the tissue. When the reverse transcription reaction continues, the extension of the first strand cDNA will include the TSO sequences, thus appending the location barcode sequences to the target sequences. In this aspect, if molecular barcode sequences are used, the molecular barcodes can be on the first strand oligo (dT) cDNA primers. In this method the cDNA synthesis is entirely done directly in the tissue section, and not in conjunction with primers attached to a array, which can allow for better penetration of the primers into the tissue section. In some aspects, both the molecular and location barcodes are on the same primer sequence.

In view of the above, it will be seen that the several advantages of the disclosure are achieved, and other advantageous results attained. The present invention can be used to detect nucleic acids other than mRNA or cDNA. In certain aspects, the in situ RNA-sequencing method can be combined with other methods of tissue analysis. For example, the in situ RNA-sequencing method can be combined with methods for labeling biomolecules with DNA aptamers or oligo-tagged antibodies, as described in U.S. Pat. No. 9,834,814. In some aspects, the sequences of the oligonucleotides attached to antibodies can be retrieved together with the mRNA sequences obtained by the method. For example, the tissue section can be stained with antibodies that have RNA oligos attached, where the oligos have a barcode sequence and 3′ poly-A tail. The barcode sequence would identify the antibody, and the location of the antibody would be provided by the released oligos from the array. In this manner, information about the gene expression (from the mRNA sequences) and the protein expression (from the oligo-linked antibodies) could be obtained together from the same tissue section, with spatial resolution. In some aspects, the antibodies can also be labeled fluorescently or chromogenically, such that IHC information could be combined with the in situ RNA sequence information. In some aspects, the in situ RNA-sequencing method can be combined with methods for acquiring DNA sequence. For example, the location barcode from the array could be attached to PCR amplicons generated in situ, such that information about genomic mutations could be obtained with spatial resolution. Comparing information from the RNA sequencing to information from the DNA sequencing could lead to insights into processes such as RNA editing or allele-specific gene expression.

In another set of aspects of the present invention, an array with fixed, non-cleavable sequences (“index oligos”) is used as a hybridization substrate to “sort” a library of oligonucleotide so that probes are hybridized to pre-determined locations on the array (FIG. 1B). In these aspects, each array feature can contain a distinct location barcode sequence, and soluble oligonucleotide probes can hybridize to the array features via the location barcodes. A major advantage of these aspects is that multiple different sequences can be captured in one array feature, as long as the sequences share the location barcode. For example, a set of oligo-dT probes could be captured on the array, wherein each oligo-dT probe also contains a unique molecular barcode. As another example, a mixture of oligo-dT probes and gene-specific cDNA primers could be captured on the array, in order to probe specific sequences in addition to polyadenylated sequences in general. In another example, PCR primers including both forward and reverse primers could be captured on the array. A combination of these or other sequences can be hybridized to the array, such that each array feature can capture a plurality of probe oligonucleotides with different sequences or functions, provided that they share the same location barcode, enabling the plurality to hybridize to the specific array feature. In this fashion, one array feature can be used to deliver a plurality of distinct probe oligonucleotides to each location within the tissue.

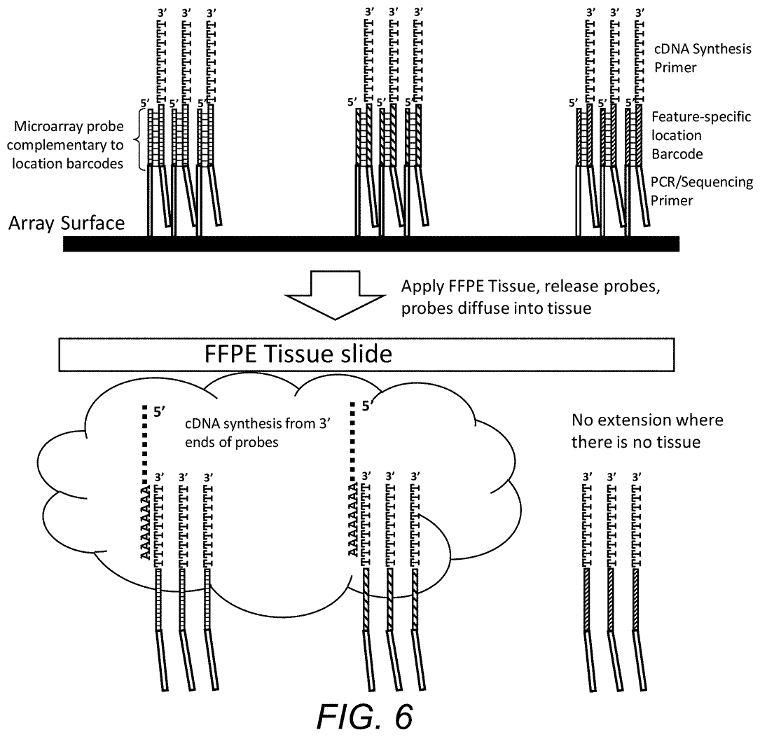

In this aspect, an array is printed where every feature contains a unique nucleotide sequence which serves as a location barcode (FIG. 6). The oligonucleotides in this array are attached to the surface (such as by their 3′ ends), and the arrayed oligonucleotides do not need to be attached with a cleavable linker. A corresponding oligonucleotide library is produced where the oligos contain a PCR primer sequence (for example at the 5′ end), as well as a sequence complimentary to the location barcodes on the array. Also included in the oligonucleotides in the probe library is another PCR primer sequence, which includes an oligo(dT) sequence at the 3′ end. If desired, the oligonucleotide probe library can be amplified by PCR prior to hybridization to the array; PCR amplification of the oligonucleotide library can yield a set of products with a PCR primer sequence at the 5′ end, location barcodes complementary to those on the array in the middle, and an oligo(dT) run at the 3′ ends.

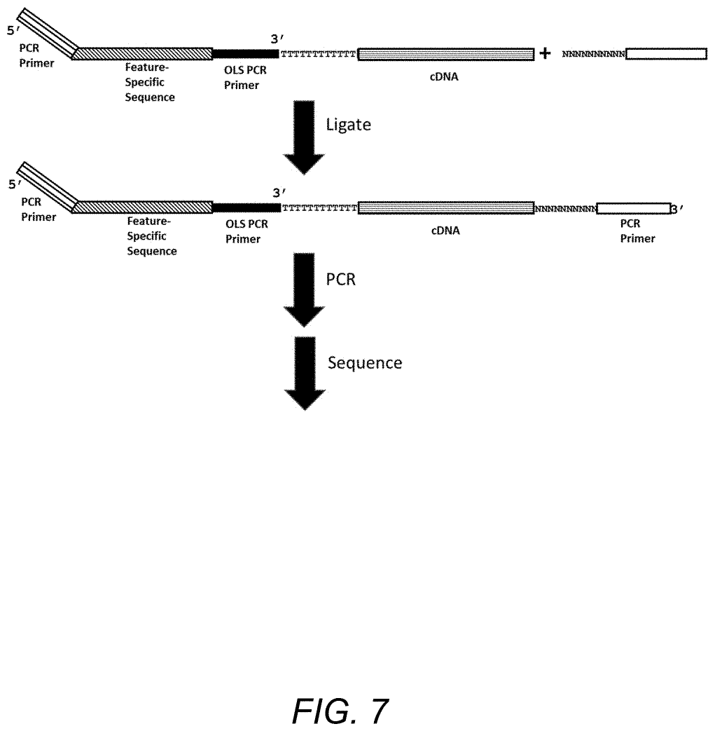

The oligonucleotide probe library is then hybridized to the array, such that each array feature captures a subset of the library containing the same location barcode. Subsequently, the tissue section to be assayed is placed above the array surface to form a “sandwich” where the oligo(dT) runs of the oligonucleotide probes hybridized to the array are then available to hybridize to the poly(A) tails of mRNAs in the tissue section (FIG. 6). After hybridization, primer extension with a reverse transcriptase will produce cDNAs primed by the oligonucleotide probe library. While the cDNAs might not extend very far, due to cross-linkage or degradation of the RNA in the tissue section, a full-length cDNA is not required to identify the mRNA being extended. After cDNA synthesis, the cDNAs primed by the oligonucleotide probe library are isolated, and a new PCR primer sequence is ligated onto the 3′ ends (FIG. 7). This primer could also contain a molecular barcode of random nucleotides (shown as N's in FIG. 7). PCR using this primer sequence plus the original 5′ end PCR primer sequence yields a cDNA library containing positional barcodes and an mRNA (cDNA) sequence on the same molecules. This library is then sequenced using typical Next-Generation sequencing (NGS) methods.

The sequencing results are then mapped back onto the array using the spatial barcodes to determine the position on the array of each cDNA sequence. A microscopic image of the tissue section can be overlaid with the sequencing results, and the positions of each RNA sequenced can be visualized against the microscopic image of the tissue. Since no cDNA is produced from features on the array that were not in contact with the tissue section, it should be straightforward to align the tissue section image with the array image. In this manner, spatial visualization of the RNA transcriptome is produced. If oligo(dT) primer sequences are used, the method should not pick up rRNAs or any other RNAs lacking a poly(A) tail. Alternatively, the entire RNA transcriptome could be assayed using a set of random-priming sequences in place of the oligo(dT) primers, or a combination of random-priming and oligo(dT) primers can be used.

The method just described could be modified to use sequence-specific primers instead of oligo(dT) priming regions on the oligonucleotide library if one wanted to look for specific mRNAs (or cDNAs). This could be done in a multiplex manner, so that a defined set of mRNAs could be assayed at the same time. In this instance, each location barcode would have a set of primers with different 3′ ends associated with it that all hybridize to the same feature. Alternatively, a mixture of oligo(dT) primers and specific primers could be used.

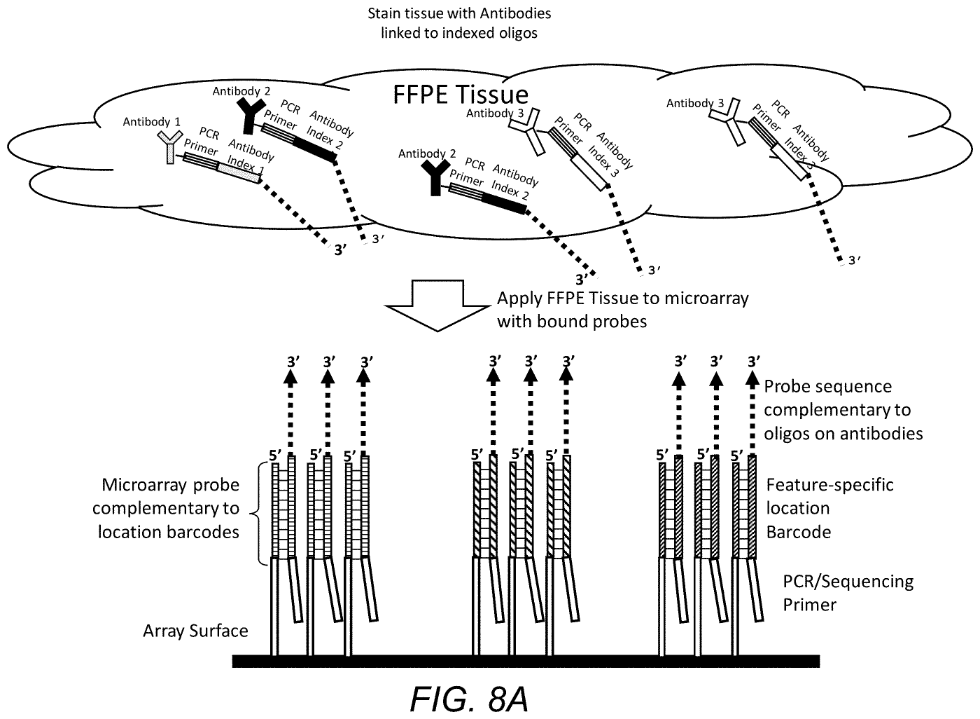

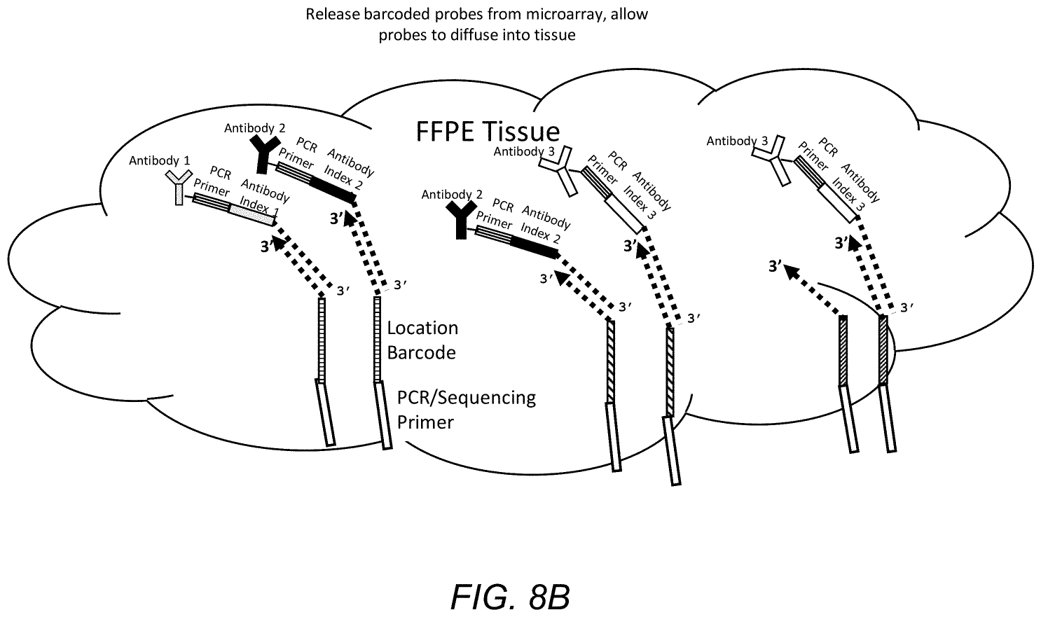

Another variation of this method could also be used to localize proteins in a tissue section (FIG. 8). In this method, the tissue section is probed using immunohistochemistry with a number of antibodies to different proteins, where each antibody has a unique oligonucleotide tag attached that is indicative of the nature of the antibody. These oligonucleotide tags contain a PCR primer region complementary to a PCR primer (for example the 3′ primer) sequence on an oligonucleotide array (there are no oligo(dT) tails on the oligonucleotides in this method); these regions hybridize to the probes after the “sandwich” is made, and primer extension using a DNA polymerase extends through the rest of the DNA attached to the antibodies, which includes an antibody-specific barcode and a 3′ PCR sequence (FIG. 8). After primer extension, the extended oligonucleotide probe library is isolated, PCR amplified using the outer PCR primer sequences, and sequenced.

Alternatively, the oligonucleotide tags attached to the antibodies could be designed such that they have a poly(A) or poly(dA) sequence at the 3′ end, enabling priming of these tag sequences by an oligo(dT) primer. In this variation, the oligonucleotide tag sequences for the antibodies should be designed so that the tag sequence is distinct from the target sequences in the sample, as the oligo(dT) primer should also capture some mRNA sequences in the sample. However, this method could enable simultaneous measurement of mRNA and protein expression in the same tissue. Again, the array feature-specific sequences can be used to identify the location of each antibody on the array surface, and this can be mapped onto the tissue section's microscopic image since no DNA is produced from regions where there is no tissue contacting the array.

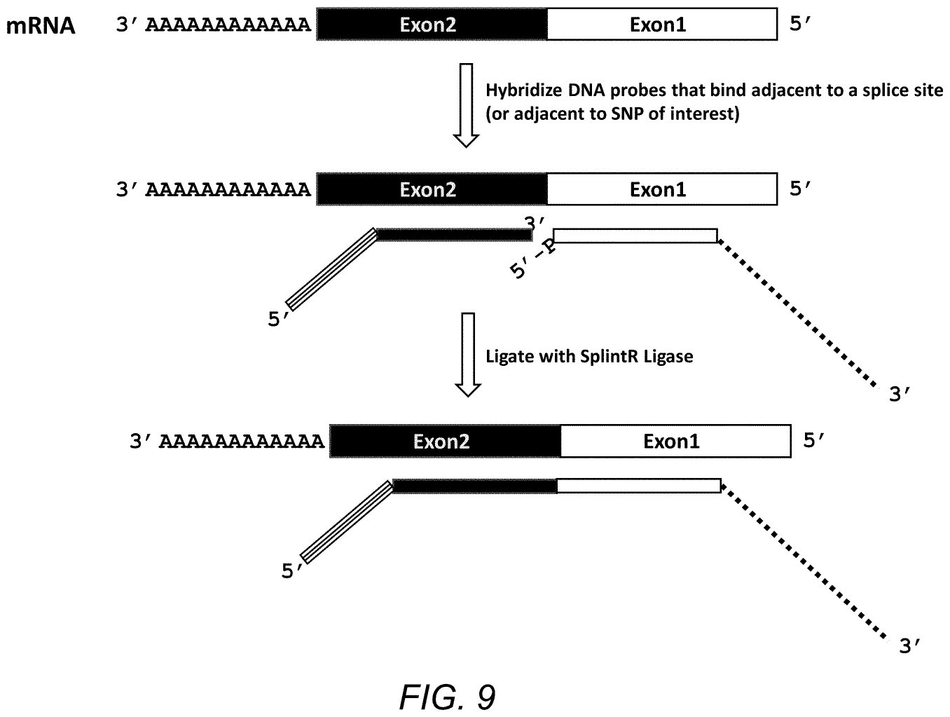

Another aspect for performing spatial analysis involves probing the sequences in the tissue with pairs of sequence-specific probes that can be ligated together. In this aspect, a collection of single-stranded DNA oligonucleotides are synthesized that will hybridize to a set of RNA transcripts to be investigated. The oligonucleotides are designed in pairs, such that each oligonucleotide pair will hybridize adjacent to one another on an RNA transcript such that the position where the two oligonucleotides lie is end-to-end on an exon-exon junction in the mature mRNA, and the probe with the 5′-end at this junction will be phosphorylated at the 5′ end (FIG. 9). After hybridization of the probes to the RNA transcripts in the tissue section, a DNA ligase is added, which ligates DNA oligonucleotides that are adjacent to one another hybridized to an RNA template, such as the SplintR DNA ligase (New England Biolabs). Use of SplintR ligase, which requires the probes to be hybridized to RNA, as well as having the probes line up at an exon-exon junction, ensures that the probes will ligate only while hybridized to the mature mRNA transcript, and not the genomic DNA. An advantage of this probe-ligation method is that it can be possible to use the hybridization probe sequences to access nucleic acids which are partially degraded or crosslinked in the tissue, as the nucleic acids in the tissue to act as a template for hybridization, rather than as a template for polymerization or a primer sequence.

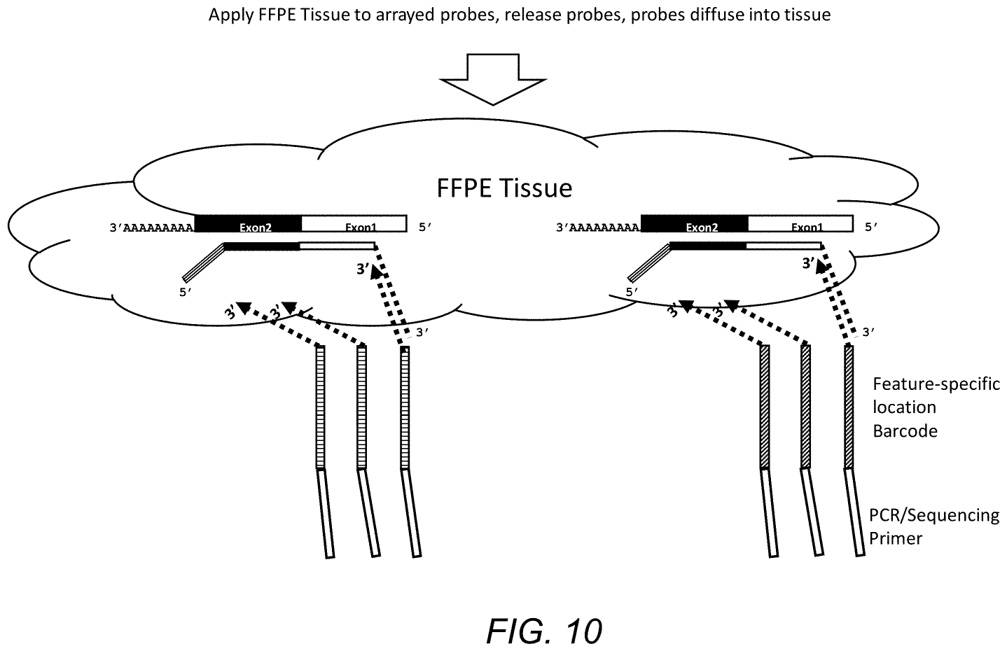

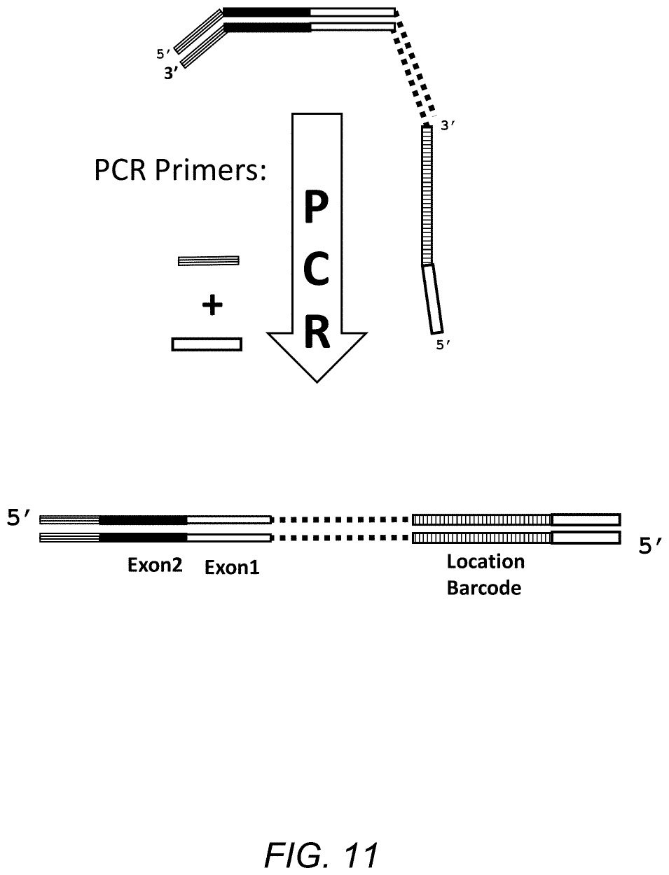

In addition to the regions complementary to the RNA, each DNA probe in the probe pairs has a region that does not hybridize to anything in the tissue section (FIG. 9). One probe has a 5′ region that will serve as a binding site for a PCR primer, while the other has a 3′ region complementary to a region of another probe set that is hybridized to a DNA array (FIG. 10). The tissue section with the probe pairs hybridized to the specific RNAs of interest is brought into contact with a DNA array where each feature on the array contains a location barcode unique to that feature, and each feature would be hybridized to oligonucleotide probes via that unique barcode. Each of these barcoded probes hybridized to the array would also contain a 3′ region which is the same on all the hybridized oligonucleotides (dashed line in FIG. 10), and which is complementary to the 3′ region of the oligonucleotides hybridized to the RNA in the tissue section. Thus, the two probes can hybridize (FIG. 10), and primer extension could be performed to extend the oligonucleotides hybridized to the array to copy the ligated RNA detection oligonucleotides. This primer extension product could then be melted off the array and tissue section (possibly with the help of RNAse to degrade the tissue RNA), and PCR using the primer sites at the ends of both strands would then be performed to amplify the ligated probe products (FIG. 11). Sequencing of the products would then be used to count the different products to determine abundance, and sequencing of the attached spatial barcodes would identify their position in the tissue. This method uses primer extension on DNA probes hybridized to RNA in the tissue, and not on the RNA itself, and thus can be more tolerant to degraded and broken RNAs which are commonly found in FFPE tissue.

In another aspect of the above method, the hybridized probes do not meet at an exon-exon boundary, since SplintR ligase should only ligate probes hybridized to RNA. Probes could be designed to meet at the site of a single nucleotide polymorphism (SNP), which would enable in situ detection of RNA SNPs, or allele-specific gene expression.

The method of ligation of two DNA probes together while hybridized to RNA in a sample has been previously described in the literature (Nucleic Acids Research 45, e128 (2017)). However, the reported method does not use SplintR ligase to differentiate oligonucleotides hybridized to RNA rather than DNA. It also does not teach having the probes meet at a splice junction to distinguish DNA from RNA hybridization, and it does not mention the use of a array, or any other method, to determine spatial location of the ligated products.

Although the foregoing invention has been described in some detail by way of illustration and example for purposes of clarity of understanding, it is readily apparent to those of ordinary skill in the art in light of the teachings of this invention that certain changes and modifications can be made thereto without departing from the spirit or scope of the appended claims. It is intended that all matter contained in the above description and shown in the accompanying drawings shall be interpreted as illustrative and not in a limiting sense.

### EXAMPLES

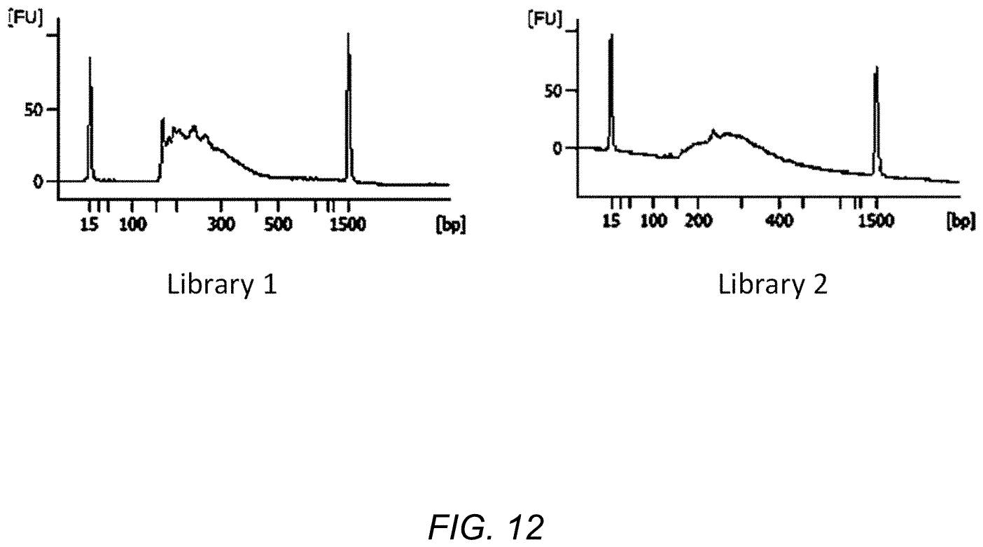

Advantageously for some aspects, we have found that PCR after first- or second-strand cDNA synthesis can be performed without first purifying the cDNA from the tissue sample. Instead the FFPE tissue containing the newly synthesized cDNA can be scraped off the slide and placed directly into a PCR tube for amplification. FIG. 12 shows two examples of Agilent Bioanalyzer traces (DNA 1000 kit) of two cDNA libraries synthesized by reverse transcription in situ on an FFPE tissue section followed by PCR on the ground up FFPE tissue containing newly synthesized first-strand cDNA. Both libraries clearly produced PCR products ranging from about 200-400 bases. Both libraries from FIG. 12 were sequenced and shown to contain cDNA copies of portions of human mRNAs. In aspects, these cDNA copies can be encoded with a spatial barcode provided from a array feature.

Using an array of spatially-barcoded released oligonucleotides, we demonstrated their ability to prime second-strand cDNA synthesis in an FFPE tissue section as outlined in FIG. 3. A human breast tumor FFPE tissue section on a microscope slide was deparaffinized, treated with pepsin, and dehydrated with alcohol. Using a first-strand cDNA oligo(dT) primer with the sequence GCAATCGTCGATAGCGTTGTTTTTTTTTTTTTTTTTTTTTTTTTTTTTTVN (SEQ ID NO. 1), a template-switching oligonucleotide (CGGCTCATCAGATTGAACACrGrGrG) (SEQ ID NO. 2), buffer, dNTPS, and reverse transcriptase, first-strand cDNA was synthesized in situ by incubating at 42C for 90 minutes, followed by 5 minutes at 85C. After cooling to room temperature and briefly washing the slide, Herculase II DNA polymerase (Agilent), polymerase buffer, and dNTPs were added to the top of the tissue, and a “sandwich” was then made with an array of released, spatially-barcoded oligonucleotides (FIG. 2). This was incubated at 72C for 2 minutes, 57C for 2 minutes, and 72C for 10 minutes. The sandwich was then broken apart and the tissue and solution were scraped into a microfuge tube, followed directly by 22 cycles of PCR using Herculase II. The PCR primers were TAGCTTGGCTATCGACACCATAAG (SEQ. ID. NO 3) and GCAATCGTCGATAGCGTTG (SEQ. ID. NO 4). Following bead purification, PCR products were amplified again to put on adapter sequences for sequencing, and the products were sequenced on a MiSeq (Illumina) using standard protocols.

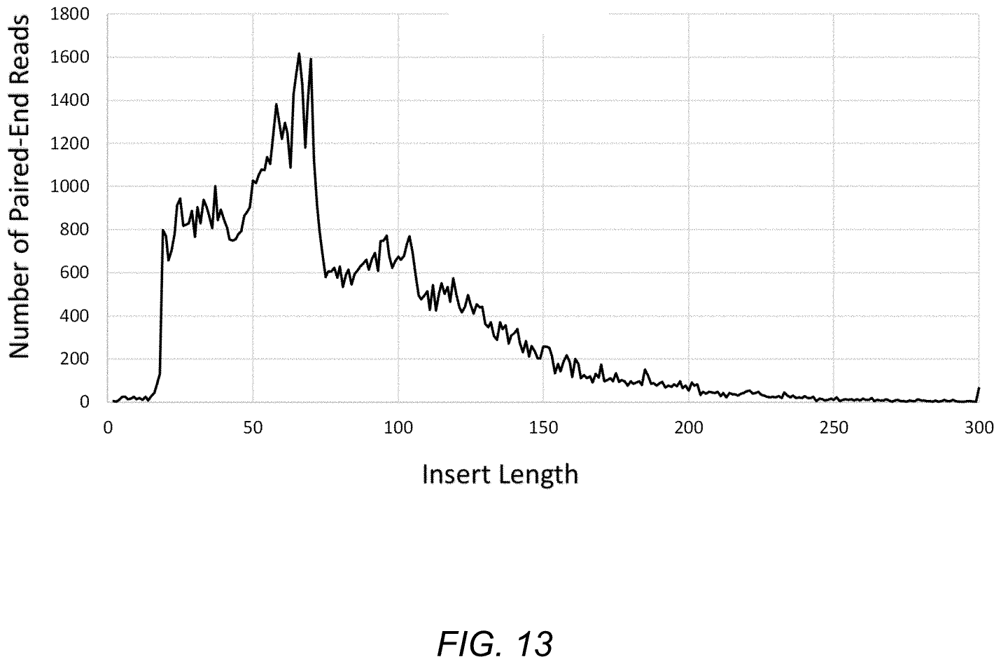

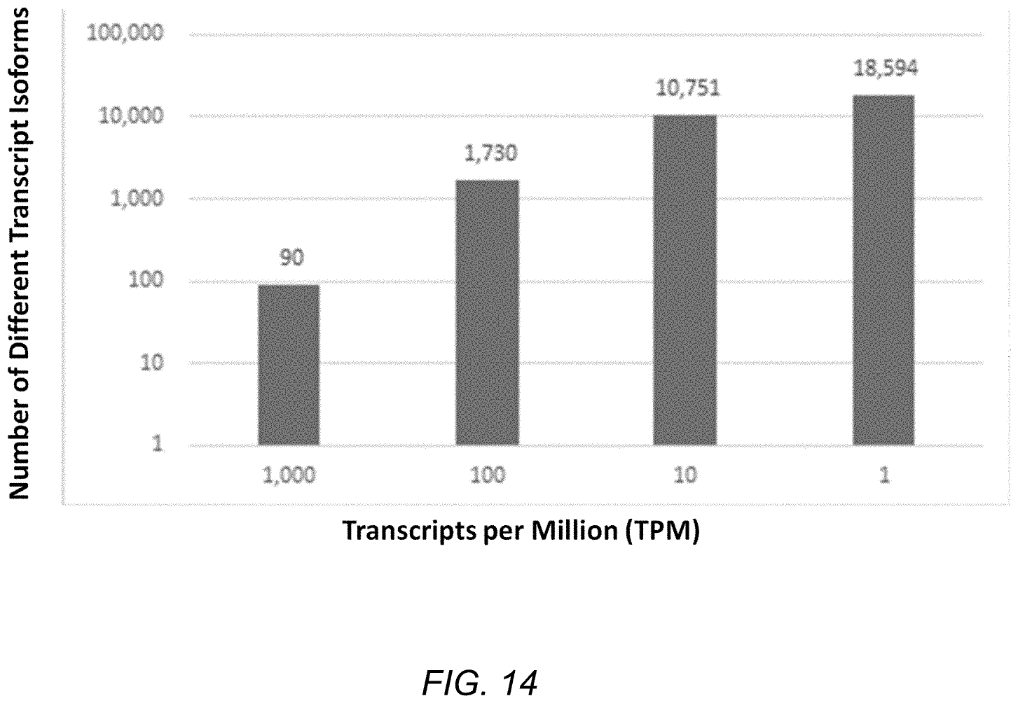

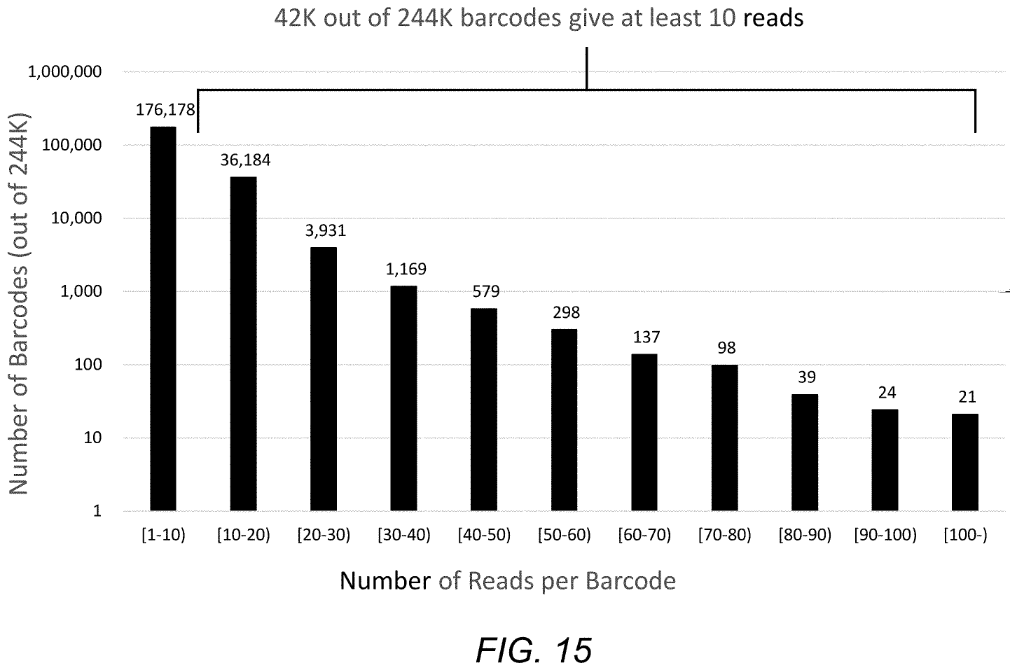

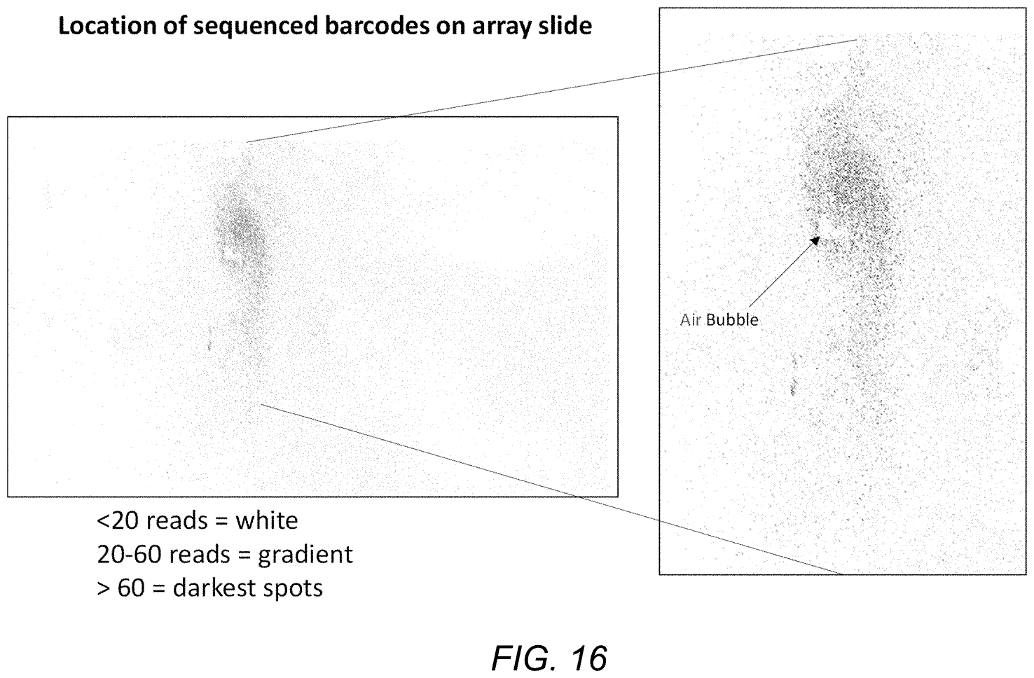

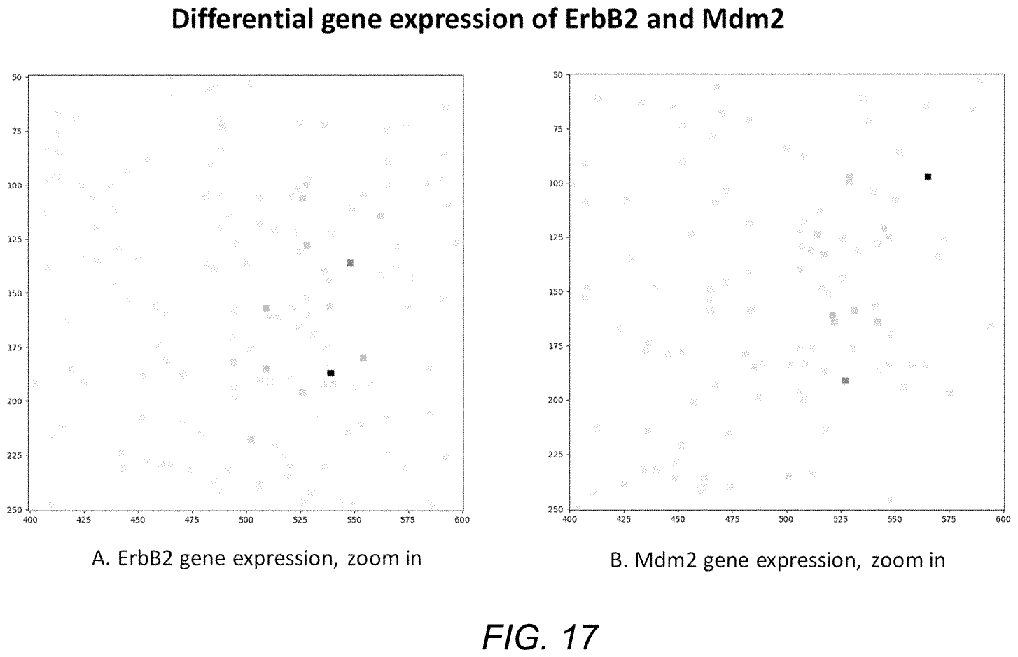

Analysis of the library thus obtained showed that a wide range of insert sizes were obtained, with the most abundant being between 50-100 bases in length and almost all smaller than 250 bases (FIG. 13). A little over 3,000 different transcript isoforms were identified, with a wide range of abundances when expressed in transcripts per million (TPM) (FIG. 14). When the spatial barcodes attached to each cDNA sequence were examined, about 42,000 of the 244,000 possible barcodes gave at least 10 reads per barcode (FIG. 15). When the location of the spatial barcodes is plotted according to the location on the array slide, with the abundance of each barcode denoted by the darkness of the spot, it is clear that most barcodes are located in the area where the slide of FFPE tissue was fixed to the slide (FIG. 16). Indeed, where there was a small air bubble atop the tissue that formed as the sandwich of the two slides was being made, the location of the air bubble can be easily seen as a dip on the number of barcodes obtained from that region (FIG. 16). In FIG. 17, the expression patterns of two representative genes from this experiment, ErbB2 and Mdm2, are shown in the same magnified area of the FFPE tissue section. The expression patterns of the two mRNAs are clearly different, indicating detection of differential spatial gene expression.

What is claimed is:

1. A method for detecting nucleic acids, the method comprising:
providing a tissue sample;
providing an array comprising a plurality of oligonucleotide probes attached to a surface of the array,
wherein each oligonucleotide probe, of the plurality of oligonucleotide probes, comprises a location barcode sequence, a primer binding sequence, and a priming sequence;
releasing the plurality of oligonucleotide probes from the array surface;
contacting the tissue sample with the released oligonucleotide probes; and
allowing the released oligonucleotide probes to diffuse into the tissue sample.

2. The method of claim 1, further comprising:
incubating the oligonucleotide probes and the tissue sample for a sufficient time to allow the plurality of oligonucleotide probes to hybridize to target nucleic acids within the tissue sample;
extending the priming sequence on the oligonucleotide probes to produce a primer extension product comprising the location barcode;
amplifying the primer extension product to result in amplified products, and
sequencing the amplified products.

3. The method of claim 1, wherein the target nucleic acids comprise mRNAs, and the priming sequence comprises oligo(dT).

4. The method of claim 1, wherein the tissue sample comprises cDNAs which each comprise at least a first-strand cDNA.

5. The method of claim 4, wherein the priming sequence binds to a sequence in the first strand cDNA.

6. The method of claim 1, wherein the tissue sample comprises cDNAs synthesized in a presence of a template switching oligonucleotide, and the priming sequence binds to a sequence added by the template switching oligonucleotide.

7. The method of claim 4, wherein the first strand cDNA comprises an adapter ligated to its 3′-end, and the priming sequence binds to the adapter.

8. The method of claim 1, wherein said plurality of oligonucleotide probes are attached to the array surface by hybridization.

9. The method of claim 8, wherein the plurality of oligonucleotide probes shares a same location barcode and are bound by an array feature comprising a complementary sequence to that location barcode.

10. The method of claim 1, wherein the plurality of oligonucleotide probes is attached to the array surface covalently.

11. The method of claim 1, wherein said plurality of oligonucleotide probes are released from the array surface by cleavage with gaseous ammonia.

12. The method of claim 1, wherein said plurality of oligonucleotide probes are released from the array surface by photocleavage.

13. The method of claim 1, wherein said plurality of oligonucleotide probes are released from the array surface by a restriction enzyme.

14. The method of claim 1, wherein said plurality of oligonucleotide probes are released from the array surface by denaturation.

15. The method of claim 1, wherein the tissue sample is contacted with the oligonucleotide probes after the oligonucleotide probes are released from the array surface.

16. The method of claim 1, wherein the tissue sample is contacted with the oligonucleotide probes before the oligonucleotide probes are released from the array surface.

17. The method of claim 1, wherein the tissue sample comprises nucleic acid tags indicative of particular antibodies.

18. A method for detecting nucleic acids, the method comprising:
providing a tissue sample;
providing a microarray that comprises a plurality of oligonucleotide probes attached to a microarray surface, wherein the oligonucleotide probes comprise a location barcode, a primer binding sequence, and a priming sequence;
releasing the plurality of oligonucleotide probes from the microarray surface while substantially maintaining their locations on the microarray surface;
contacting the tissue sample with the oligonucleotide probes;
allowing the oligonucleotide probes to diffuse into the tissue sample, and incubating the oligonucleotide probes and the tissue sample for a sufficient time to allow the plurality of oligonucleotide probes to hybridize to target nucleic acids within the tissue sample;
extending the priming sequence on the oligonucleotide probes to produce a primer extension product comprising the location barcode;
amplifying the primer extension product to result in amplified products, and
sequencing the amplified products.


SEQUENCE LISTING (4 sequences)

SEQ ID NO: 1 - DNA, 51 bp, Artificial Sequence
  Synthetic
  misc_feature (51)..(51): n is a, c, g, or t
gcaatcgtcgatagcgttgttttttttttttttttttttttttttttttvn

SEQ ID NO: 2 - DNA, 23 bp, Artificial Sequence
  Synthetic
cggctcatcagattgaacacggg

SEQ ID NO: 3 - DNA, 24 bp, Artificial Sequence
  Synthetic
tagcttggctatcgacaccataag

SEQ ID NO: 4 - DNA, 19 bp, Artificial Sequence
  Synthetic
gcaatcgtcgatagcgttg